(19) United States
(12) Patent Application Publication
(10) Pub. No.: US 2016/0150973 A1
(43) Pub. Date: Jun. 2, 2016

(54) SUBJECT INFORMATION ACQUISITION APPARATUS

(71) Applicant: CANON KABUSHIKI KAISHA, Tokyo (JP)

(72) Inventor: Hiroshi Abe, Kyoto-shi (JP)

(21) Appl. No.: 14/950,499

(22) Filed: Nov. 24, 2015

(30) Foreign Application Priority Data
Nov. 28, 2014 (JP) 2014-242447

(51) Int. Cl. A61B 5/00; A61B 5/05

(52) U.S. Cl. CPC A61B 5/0095; A61B 5/05; A61B 5/7271; A61B 5/0035

(57) ABSTRACT

A subject information acquisition apparatus includes receiving units that are arranged along an arc shape and configured to receive a photoacoustic wave generated in a subject in response to light from a light source and convert the photoacoustic wave into a time-series electric signal, a driving unit configured to scan the receiving units, and a processing unit configured to acquire information about an inside of the subject. The light source emits light at a certain timing. The receiving unit receives a photoacoustic wave at a correspondent timing synchronized with light emission. The driving unit causes the receiving unit to perform scanning to allow the receiving unit to receive the photoacoustic wave in a predetermined region in synchronization with the correspondent timing. The processing unit acquires the information based on positional coordinates in the region and a signal resulting from summation of time-series electric signals corresponding to the correspondent timing.

### BACKGROUND OF THE INVENTION

1. Field of the Invention

The present disclosure generally relates to a subject information acquisition apparatus for acquiring information on characteristics of a subject.

2. Description of the Related Art

In medical fields, researches on optical imaging are proceeding in which a light source irradiates a subject with light, information about the inside of the subject is obtained on the basis of a response to the light, and the information is visualized. One of techniques for this optical imaging is photoacoustic imaging. In photoacoustic imaging, a tissue generates an elastic wave after absorbing the energy of pulsed light that has propagated and diffused in a subject, the generated elastic wave is received as a signal, and information about the internal characteristics of the subject is visualized on the basis of the received signal.

When a subject is irradiated by light, an inspection target portion (for example, a tumor in a living body), which has a higher rate of light energy absorption than the other tissues, absorbs light energy and instantaneously expands to generate an elastic wave. An acoustic receiver receives this elastic wave as a signal. The signal is analyzed. Consequently, characteristic information in photoacoustic imaging is obtained. The characteristic information is the distribution of optical characteristic values such as the distribution of initial sound pressures, the distribution of light absorption energy densities, or the distribution of light absorption coefficients. By measuring such information using lights with a plurality of wavelengths, the information can also be used for quantitative measurement of specific substances (hemoglobin concentration in blood, oxygen saturation in blood, etc.) in the subject (see “Photoacoustic Tomography: In Vivo Imaging From Organelles to Organs, Lihong V. Wang Song Hu, Science 335, 1458-1462 (2012)”).

For the calculation of such characteristic information, the following two methods are used. One method is a microscopic method of reconstructing a multidimensional image using pieces of data at many points where envelope detection is performed upon an elastic wave transmitted from beneath the creepage surface of a single acoustic receiver. The other method is a tomography method of reconstructing a multidimensional image on the basis of signals of elastic waves that have been three-dimensionally generated in a subject and then been received by acoustic receivers disposed at many points.

### SUMMARY OF THE INVENTION

In the microscopic (scan) method, a high-contrast and high-resolution image (focused image) can be obtained by scanning many points one by one. In a case where the image of a broad area is generated, the acquisition of data at each point takes time. In contrast, in the tomography method of reconstructing an image after receiving pieces of data at many points at a time, a measurement time can be shortened. However, according to the arrangement of acoustic receivers, the lack of data and noise such as a reconstruction artifact may occur. This leads to the reduction in the viewability of an image.

The present disclosure provides a subject information acquisition apparatus that includes a light source, a plurality of receiving units that are arranged along an arc shape and are configured to receive a photoacoustic wave generated in a subject in response to light irradiation from the light source to the subject and convert the photoacoustic wave into a time-series electric signal, a driving unit configured to scan the plurality of receiving units, and a processing unit configured to acquire characteristic information about an inside of the subject. The light source emits light at a certain timing. The receiving unit receives a photoacoustic wave at a correspondent timing synchronized with emission of light. The driving unit causes the receiving unit to perform scanning so as to allow the receiving unit to receive the photoacoustic wave in a predetermined region in synchronization with the correspondent timing. The processing unit acquires the characteristic information based on positional coordinates in the region and a signal resulting from summation of time-series electric signals corresponding one-to-one to the correspondent timing.

Further features of the present disclosure will become apparent from the following description of exemplary embodiments with reference to the attached drawings.

### BRIEF DESCRIPTION OF THE DRAWINGS

FIG. 1 is a schematic diagram illustrating a subject information acquisition apparatus according to an embodiment of the present disclosure.

FIG. 2 is a flowchart illustrating the process of a subject information acquisition apparatus according to an embodiment of the present disclosure.

FIG. 3 is a schematic diagram describing an acoustic receiver according to an embodiment of the present disclosure.

FIG. 4 is a schematic diagram illustrating a display screen for a subject information acquisition method according to an embodiment of the present disclosure.

FIG. 5 is a flowchart illustrating the process of a subject information acquisition apparatus according to a first embodiment of the present disclosure.

FIGS. 6A and 6B are schematic diagrams illustrating display screens in a subject information acquisition apparatus according to the first embodiment.

FIG. 7 is a flowchart illustrating the process of a subject information acquisition apparatus according to a second embodiment of the present disclosure.

FIG. 8 is a schematic diagram describing an acoustic receiver according to the second embodiment.

FIG. 9 is a schematic diagram illustrating a display screen in a subject information acquisition apparatus according to a third embodiment of the present disclosure.

FIG. 10 is a schematic diagram describing an acoustic receiver according to the third embodiment.

FIG. 11 is a flowchart illustrating the process of a subject information acquisition apparatus according to the third embodiment.

FIGS. 12A to 12C are schematic diagrams illustrating display screens in a subject information acquisition apparatus according to the third embodiment.

### DESCRIPTION OF THE EMBODIMENTS

Preferred embodiments of the present disclosure will be described below with reference to the accompanying drawings. Here, it is to be noted that the sizes, materials, shapes and relative arrangements of components described below should be changed as appropriate in accordance with the configuration of an apparatus according to an embodiment of the present disclosure or various conditions, and are not intended to limit the scope of the present disclosure to the following descriptions.

A subject information acquisition apparatus according to an embodiment of the present disclosure is an apparatus that uses a photoacoustic effect to irradiate a subject with light (an electromagnetic wave), receive an acoustic wave generated and transmitted in the subject, and acquire information about the internal characteristics of the subject as image data. A subject information acquisition apparatus according to an embodiment of the present disclosure has a function of transmitting an ultrasound wave and a function of receiving a reflected wave (echo wave) from the inside of a subject and can acquire characteristic information from the reflected wave as image data.

The characteristic information indicates the distribution of sources of acoustic waves generated in response to light irradiation, the distribution of concentrations of substances forming a tissue, the distribution of initial sound pressures in the subject, or the distribution of light energy absorption densities or light energy absorption coefficients derived from the initial sound pressure distribution. The substances forming a tissue are, for example, blood components represented by the distribution of oxygen saturations or the distribution of concentrations of oxyhemoglobins or deoxyhemoglobins, fat, collagen, and moisture. Furthermore, information about the distribution of acoustic impedances in the subject which is obtained by performing known information processing upon electric signals based on reflected waves may also be regarded as a kind of the characteristic information.

An acoustic wave in the present disclosure is typically an ultrasound wave, and includes an elastic wave called sound wave or acoustic wave. An acoustic wave generated in the photoacoustic effect is referred to as a photoacoustic wave or an optical ultrasound wave. In the following, among acoustic waves, an acoustic wave transmitted toward the inside of a subject by an acoustic receiver and an acoustic wave reflected in the subject after being transmitted are referred to as “ultrasound waves”. Among acoustic waves, an acoustic wave generated in a subject in response to light irradiation is referred to as a “photoacoustic wave”. It is noted that these definitions are performed for the convenience of the distinction between acoustic waves and do not limit the wavelengths of the acoustic waves.

In the present disclosure, a plurality of photoacoustic waves are chronologically sampled while changing the direction and position of an acoustic receiver with respect to a subject and image reconstruction is performed using pieces of time-series sampling data. It is desirable that a probe include two or more acoustic receivers and have one or more focused spot. Pieces of data of photoacoustic waves received by a group of acoustic receivers having a common focused spot are added up. Envelope detection is performed to calculate an amplitude value in the vicinity of the focused spot from data around a sampling point which is estimated on the basis of a curvature and the velocity of sound. By performing this processing for each piece of acquired data, characteristics distribution image data can be obtained. By adding up received photoacoustic signals that are obtained at a single measurement point by many acoustic receivers, these signals can be combined as a pseudo high-aperture-ratio received signal.

Thus, using photoacoustic waves received by many acoustic receivers, data in the vicinity of a focused spot can be calculated and characteristics distribution image data can be generated. If pieces of data at a plurality of focused spots can be acquired, the characteristics distribution image data can be rapidly obtained. If acoustic receivers are placed to have large visual angles from a focused spot in a probe, the probe can always receive a signal at the focused spot. This can prevent the lack of data in the process of estimation of tomography image data and improve the viewability of a tomography image.

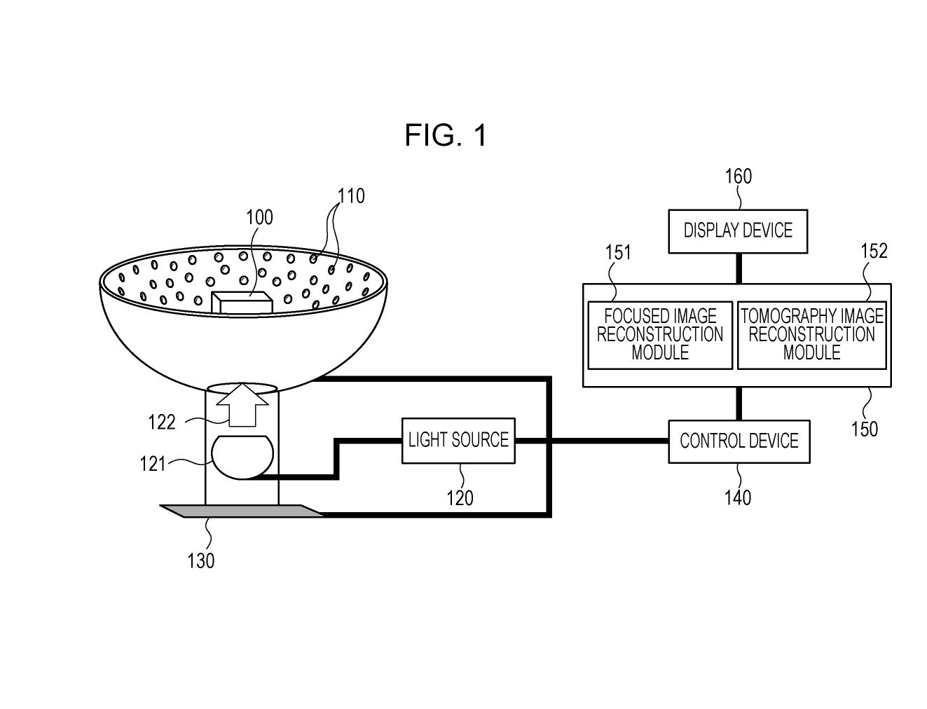

A subject information acquisition apparatus according to an embodiment of the present disclosure will be described with reference to FIG. 1. Although a subject 100 is not a part of a subject information acquisition apparatus and is an information acquisition target, examples of the subject 100 will be described. Major uses of a subject information acquisition apparatus according to an embodiment of the present disclosure are diagnosis of, for example, a malignant tumor or a vascular disease of a person or an animal and follow-up of chemical treatment. The subject 100 is therefore a living body, and, more specifically, is a diagnosis target part such as the breast, neck, abdomen, arm, foot, or hand of a human body or an animal. A light absorber in a subject represents a part having a relatively high absorption coefficient in the subject. For example, in a case where a measurement target is a human body, oxyhemoglobin, deoxyhemoglobin, a blood vessel including a large amount of oxyhemoglobin or deoxyhemoglobin, or a malignant tumor including a large number of newborn blood vessels is the light absorber. Besides, for example, plaque of the carotid artery wall is a light absorber.

Each component in a subject information acquisition apparatus will be described below.

### (Light Source)

A light source 120 is preferably a pulsed light source capable of generating pulsed light (122) on the order of several nanoseconds to several microseconds. More specifically, in order to efficiently generate a photoacoustic wave, a pulse width of approximately 10 nanoseconds is used. As the light source 120, a light-emitting diode can be used instead of a laser. Examples of the laser include solid-state laser, a gas laser, a dye laser, and a semiconductor laser. Emitted light preferably has a wavelength with which the light reaches the inside of a subject, and, more specifically, a wavelength ranging from 500 nm to 1200 nm in a case where the subject is a living body.

### (Optical System)

Light emitted from the light source 120 is guided to a subject while being processed into a desired light distribution shape by optical components such as a lens and a mirror. It is also possible to propagate the light using, for example, a light guide such as an optical fiber. An optical system 121 includes, for example, a mirror that reflects light, a lens that collects and expands light and changes the shape of light, and a diffuser that diffuses light. It is desirable that light not be collected by a lens and be spread over a certain area from the viewpoint of safety for a living body and expansion of a diagnosis region.

### (Acoustic Receiver)

An acoustic receiver 110 (receiving unit) receives acoustic waves (a photoacoustic wave and an echo wave) and converts such an acoustic wave into an analog electric signal. An acoustic receiver that makes use of a piezoelectric phenomenon, resonance of light, or a change in capacitance can be used. The acoustic receiver 110 is usually provided in a form of a probe in which a transducer is held in a housing. In this specification, the acoustic receiver is also referred to as a probe. As used herein, the term “unit” generally refers to any combination of software, firmware, hardware, or other component, such as circuitry, that is used to effectuate a purpose.

A plurality of transducers for transmitting and receiving acoustic waves do not necessarily have to be arranged along a line, and may be arranged along a plurality of lines as called a 1 D array, a 1.5 D array, a 1.75 D array, and a 2 D array. Furthermore, by arranging the transducers in an arc shape, one or more focused spots can be obtained. A plurality of transducers may be disposed in a spherical-shell supporting member. More specifically, as disclosed in International Publication No. 2010/030817, a plurality of transducers may be disposed so that the reception surfaces of the transducers are three-dimensionally arranged along a spiral fashion on the inner surface of a bowl-shaped supporting member. Thus, by arranging a plurality of transducers in an array, it is possible to perform the calculation of a tomography image, which cannot be performed with only data of a single transducer, the discrimination among pieces of signal data at a plurality of focused spots, and the correction of each signal. Between the interface of a subject and the reception surfaces of the transducers arranged on the inner side of a bowl, water or a gel material having an acoustic impedance close to that of water may be provided. As a result, it is possible to easily operate the acoustic receiver 110 and measure an acoustic wave regardless of the shape of a measurement target.

The acoustic receiver 110 preferably has a function of a transmitter for transmitting an ultrasound wave to the subject 100 and a function of a receiver for receiving an echo wave that has propagated through the inside of the subject 100. This enables signal detection in the same region and space saving. The transmitter and the receiver may be separately provided. A receiver for a photoacoustic wave and a receiver for an echo wave may be separately provided. The acoustic receiver 110 may be mechanically moved or may be a hand-held acoustic receiver manually operated by a user.

### (Probe Driving Unit)

A probe driving unit 130 (driving unit) can cause the acoustic receiver 110 to scan a subject and change the position of the acoustic receiver 110 in predetermined three-dimensional space. The probe driving unit 130 is, for example, a stepping motor or a piezoelectric element. A subject information acquisition apparatus can make a swinging instruction for an operator in the form of sound or screen display. In the case of a hand-held probe, an operator can manually change a position. In this case, a positional information acquisition mechanism is achieved by providing a sensor for checking the distance of scanning performed by an operator and the angle of a probe. Data obtained by the sensor may be estimated by using information at the time of detection of a signal performed by the acoustic receiver 110 or performed in response to a trigger as supplementary information. The sensor is, for example, a magnetic sensor, an infrared sensor, an angular sensor, or an acceleration sensor. A hand-held probe is preferable in that it can easily reflect an operator's intention at the time of determination of a measurement region.

### (Control Device)

A control device 140 performs amplification processing and digital conversion processing upon an analog electric signal output from the acoustic receiver 110. The control device 140 includes, for example, an amplifier, an analog to digital (A/D) converter, a field programmable gate array (FPGA) chip, and a central processing unit (CPU). In a case where the acoustic receiver 110 outputs a plurality of signals received from a plurality of transducers, the acoustic receiver 110 can output the summation of these signals. Alternatively, setting may be changed so that these received signals are separately output.

In the present disclosure, a photoacoustic wave signal is a concept including a time-series analog electric signal output from the acoustic receiver 110 and a time-series signal processed by the control device 140. An ultrasound wave signal is a concept including a time-series analog electric signal output from the acoustic receiver 110 that has received an echo wave and a time-series signal obtained after processing in the control device 140.

The control device 140 controls the timing of emission of pulsed light and the timings of transmission and reception of an electric signal which are triggered by the pulsed light. The control device 140 controls the probe driving unit. More specifically, the control device 140 starts or stops the operation of a motor and performs position control at the time of the reception of a photoacoustic wave signal. Furthermore, the control device 140 can also set an instruction for a positional information signal at the time of measurement.

### (Signal Processing Device)

A signal processing device 150 generates information about the inside of a subject on the basis of a digital signal. As the signal processing device 150, an information processing device such as a workstation is used. Correction processing, image reconstruction processing, and the like to be described later are performed by software programmed in advance. The software includes a focused image reconstruction module 151 that is characteristic processing in the present disclosure and a tomography image reconstruction module 152. These modules may be provided independently from the signal processing device 150. The signal processing device 150 can perform signal processing in all of one-dimensional space, two-dimensional space, and three-dimensional space.

In photoacoustic imaging, the focused image reconstruction module 151 can calculate characteristic information in the vicinity of a focused spot in a living body with a signal that is a result of summation of photoacoustic wave signals obtained by the acoustic receivers 110 among probes having focused spots. The probe driving unit 130 moves the acoustic receivers 110, obtains pieces of characteristic information at a plurality of coordinates, and perform mapping of these pieces of characteristic information, so that a focused image (characteristic information) can be obtained.

The tomography image reconstruction module 152 performs image reconstruction with photoacoustic wave signals to form a tomography image (characteristic information). The image reconstruction is processing for allocating (projecting) an arbitrarily extracted reception signal (or a projection signal obtained by arbitrarily weighting a reception signal) to each reconstruction pixel (voxel).

Characteristic information such as the distribution of acoustic impedances of a subject is generated using ultrasound wave signals. As an image reconstruction algorithm, a method known in the tomography technique is used. The method is, for example, back projection in a time domain or a Fourier domain or phasing addition (delay and sum). When a long time can be used for reconstruction, an image reconstruction method such as an inverse problem analysis method achieved by repetition processing may be used. The tomography image reconstruction module 152 performs, upon the ultrasound wave signal, delay addition processing for phase matching and addition processing subsequent to the delay addition processing. Consequently, it is possible to form characteristic information such as an acoustic impedance in a subject and speckle pattern data acquired through scattering in the subject.

Each of the focused image reconstruction module 151 and the tomography image reconstruction module 152 includes a device such as a CPU or a GPU and a circuit such as an FPGA or an ASIC. The control device 140 and the signal processing device 150 are sometimes integrated. In this case, characteristic information such as the acoustic impedance of a subject and the distribution of optical characteristic values can be generated in hardware processing rather than in software processing performed by a workstation. The control device 140 and the signal processing device 150 correspond to a processing unit according to an embodiment of the present disclosure.

### (Display Device)

A display device 160 (display unit) displays characteristic information such as the distribution of optical characteristic values output from the signal processing device 150. As the display device 160, for example, a liquid crystal display, a plasma display, an organic EL display, an FED, a spectacle-type display, or a head-mount display can be used. The display device 160 can be provided separately from a main body of the subject information acquisition apparatus. In this case, acquired information about a subject may be transmitted to the display device 160 in a wired or wireless manner.

### (Processing Flow)

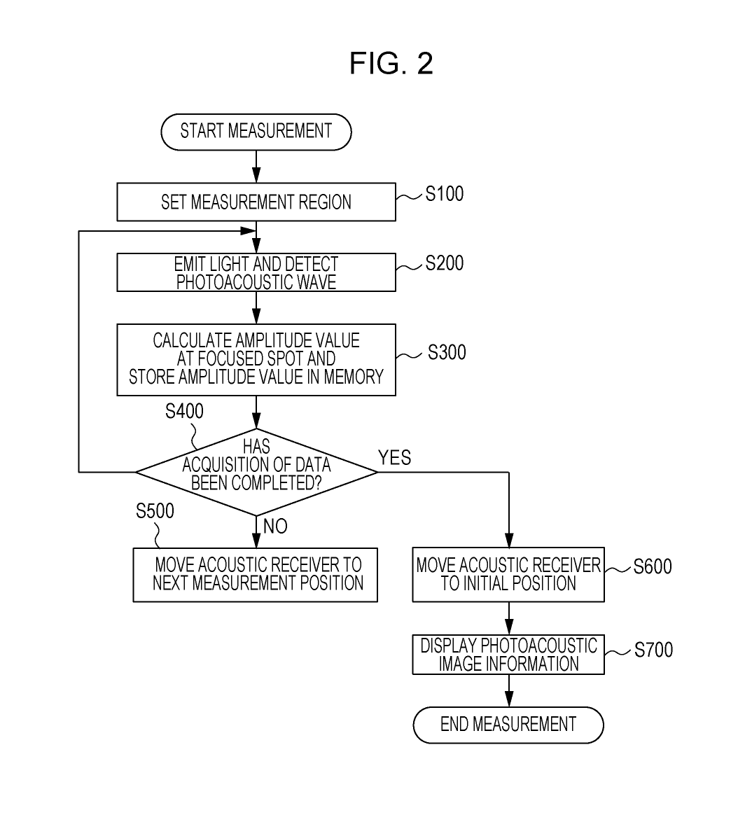

Next, a subject information acquisition method performed by a subject information acquisition apparatus will be described with reference to FIG. 2. The control device 140 reads out a program describing the subject information acquisition method from a memory in the control device and controls the operation of each component in the subject information acquisition apparatus, so that the following process is performed.

### (Step S100: Measurement Area Determination Processing)

In this processing, the probe driving unit 130 specifies a region where the acoustic receiver 110 performs scanning. The scanning region may be specified on the basis of absolute coordinates at which the probe driving unit 130 can cause the acoustic receiver 110 to perform scanning. Alternatively, the scanning region may be specified on the basis of information about a subject obtained in advance by another diagnostic imaging apparatus or information obtained as a result of observation of the shape or internal condition of a subject in the scanning region. The information about the shape of a subject can be obtained from an image captured by, for example, a digital camera or a video camera. The information about the internal condition of a subject can be obtained from a photoacoustic image, a ultrasound wave image, an X-ray CT image, an MRI image, or a PET image. In photoacoustic imaging or ultrasound wave imaging, a subject information acquisition apparatus is more preferable in that it can have the above-described image capturing function and the commonality of components can be achieved.

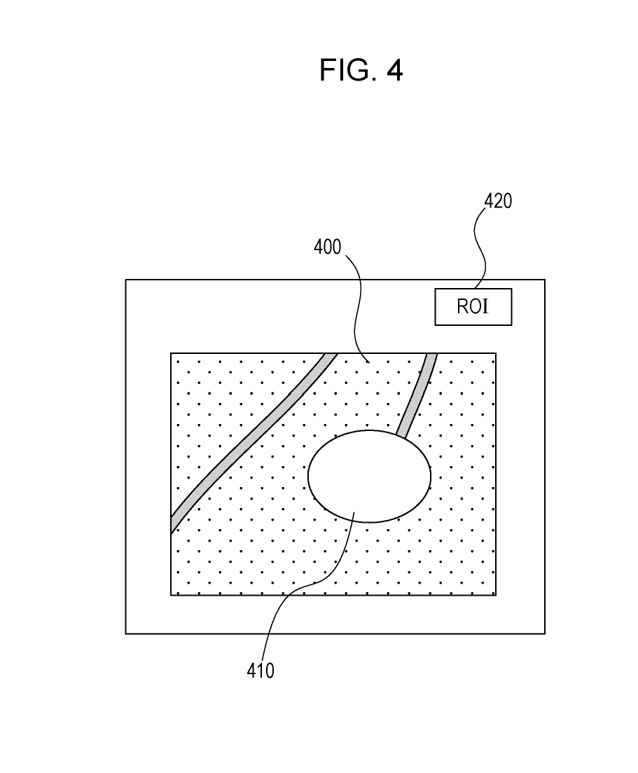

It is desirable that a subject information image be displayed on the display device 160 so as to allow an operator to optionally select a region of interest (ROI) in the image. For example, when a photoacoustic image 400 that has been obtained in advance with a tomography method and is placed in a scanning region is displayed on the display device 160 as illustrated in FIG. 4, it is possible to, with an input device such as a mouse or a touchscreen, set a scanning region selection icon 420 and allow an operator to enclose an indistinct region 410 in a box with the mouse or set a scanning region by movement of, for example, a fingertip on the touchscreen. It is desirable that the position of an origin be calibrated with a calibration table for an apparatus or by an external apparatus. However, an origin may be set at the start time of measurement and the coordinates of a characteristic image calculation after the measurement may be changed.

### (Step S200: Photoacoustic Wave Signal Acquisition Processing)

In this processing, the acoustic receiver 110 receives a photoacoustic wave generated in a subject and generates a photoacoustic wave signal.

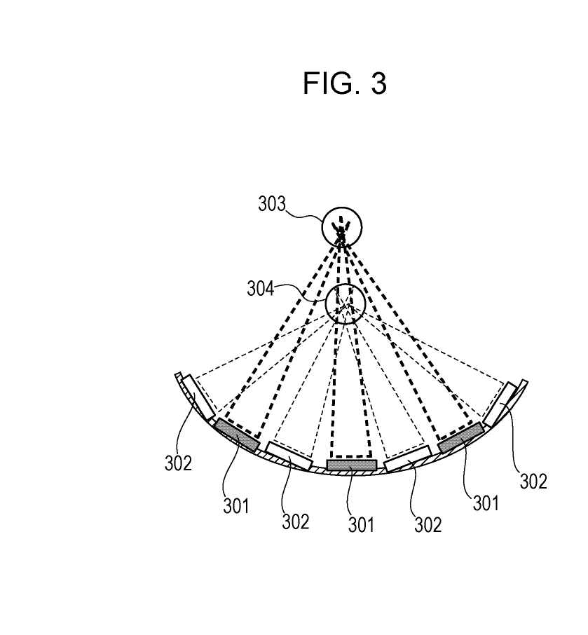

First, the subject 100 is irradiated by the pulsed light 122 emitted from the light source 120 via the optical system 121. The pulsed light 122 is absorbed by a light absorber in the subject 100, so that a photoacoustic wave is generated. The control device 140 causes a plurality of transducers in the acoustic receiver 110 to start the reception of a photoacoustic wave in synchronization with the emission of the pulsed light. A photoacoustic wave signal output from the acoustic receiver 110 is processed in the control device 140 and is stored in a memory. At that time, the control device 140 performs the summation of a plurality of photoacoustic wave signal for each focused spot and outputs a resultant signal. The description of a focused spot will be made with reference to FIG. 3. An acoustic receiver group 301 and an acoustic receiver group 302 are placed on corresponding surfaces of a probe which have different curvatures. A focused spot represents a region where acoustic reception areas of acoustic receivers in an acoustic receiver group overlap. The focused spot of the acoustic receiver group 301 is a region 303, and the focused spot of the acoustic receiver group 302 is a region 304. Thus, referring to FIG. 3, there are two focused spots. In this embodiment, this processing does not necessarily have to be performed once, and may be performed a plurality of times.

### (Step S300: Focused Spot Amplitude Value Calculation Processing)

In this processing, the focused image reconstruction module 151 acquires data on an amplitude value in the vicinity of a focused spot corresponding to photoacoustic wave signals the summation of which is performed. A photoacoustic wave travels from a signal generation point in the subject 100 as a spherical wave. Photoacoustic wave signals corresponding to a focused spot, the summation of which is performed, are received by transducers at the same distance (spatial distance) from the focused spot. Accordingly, in a case where a photoacoustic wave signal is generated near the focused spot, in-phase signals can be obtained around there. When each transducer is placed on the radius of curvature R [m] and the propagation speed of a photoacoustic wave in a living body is c [m/s], a time τ taken for the propagation of a signal from a focused spot to a transducer can be calculated by τ=R/c. In the case of common living bodies, the propagation speed of 1540 [m/s] is used as the propagation speed c. Accordingly, the amplitude value of photoacoustic wave signals, the summation of which is performed, at a time τ or an amplitude value at a τ·fs-th sampling point when a sampling frequency is fs [Hz] is calculated as an amplitude value at the focused spot. In order to reduce the effect of waveform distortion of a reception signal in the frequency band of a transducer, a signal resulting from the summation of photoacoustic wave signals may be subjected to envelope detection. A calculated value is stored in a focused spot positional coordinate index in a memory. In a case where there are multiple focused spots, an amplitude value is calculated for each of them in accordance with a corresponding radius of curvature R[m] and is stored in a memory.

The number of focused spots is not limited to one. According to the arrangement of the transducers, a plurality of highly-focused regions may be additionally calculated. At that time, in a case where data is stored in an index in which data previously obtained at a measurement point has already been stored, one of a method of storing an average of the stored data and calculated data and a method of overwriting the stored data with calculated data, which has been set in advance, is performed.

In a case where an operator manually moves a hand-held probe and does not use the probe driving unit 130, positional information of the acoustic receiver 110 is calculated to acquire an index (positional coordinates) required for the storage of data in a memory. In order to calculate a relative positional coordinates, for example, a magnetic sensor is disposed for the acoustic receiver 110 and the positional coordinates of the acoustic receiver 110 are calculated on the basis of a magnetic variation. Alternatively, an optical sensor such as an optical trackball may be disposed for the acoustic receiver 110 and the positional coordinates of the acoustic receiver 110 may be calculated using infrared measurement data. In step S200, the order of calculations may be changed so that an amplitude value is calculated from a signal stored for each transducer and then the summation of the amplitude values is performed. When the summation of photoacoustic wave signals is performed for each focused spot, the velocity of sound in a subject may not be constant. In this case, before the summation, sound velocity components to be transferred to corresponding transducers may be adjusted so that the phases of signal components received from a specific focused spot are aligned. Alternatively, the velocity of sound at which the phases of signal components received from a focused spot are aligned may be calculated on the assumption that the velocity of sound for the transducers is constant. In order to determine the degree of alignment of phases, for example, a Coherent Factor (CF) used for the evaluation of variations in signals is used. A velocity of sound at which the large value of CF is obtained may be employed.

### (Step S400: Processing for Determining the Number of Repetitions of Steps S200 and S300)

In this processing, the control device 140 determines whether the number of times of acquisition of a photoacoustic wave signal in steps S200 and S300 reaches a predetermined number. When the number of repetitions does not reach the predetermined number, the process from step S200 to step S300 is repeated. The number of repetitions is calculated on the basis of the scanning pitch of the probe driving unit 130 set in advance. The scanning pitch may be input from an input device such as a touch panel or keyboard by an operator. In the case of a hand-held type, the determination of the number of repetitions may be performed while performing measurement. For example, while an externally disposed button is pressed, measurement is repeated. Alternatively, a contact sensor may be disposed for a hand-held probe and measurement may be performed while an operator brings the hand-held probe into contact with a subject.

### (Step S500: Processing for Moving Acoustic Receiver to the Next Measurement Position)

In this processing, the probe driving unit 130 moves the acoustic receiver 110 to a measurement position in a measurement area set in step S100. After completion of the movement, the processing of step S200 may be repeated. Alternatively, in a case where the amount of movement of the acoustic receiver 110 and the acceleration of the acoustic receiver 110 are small, the process subsequent to step S200 may be performed while moving the acoustic receiver 110.

### (Step S600: Processing for Moving Acoustic Receiver to Initial Position)

In this processing, the probe driving unit 130 moves the acoustic receiver 110 to a predetermined position of an origin.

### (Step S700: Photoacoustic Image Information Display Processing)

In this processing, the focused image reconstruction module 151 displays photoacoustic wave amplitude data stored in a memory on the display device 160 as an image. In a case where the amplitude data is three-dimensional data, image may be three-dimensionally displayed by rendering or maximum intensity projection (MIP) images that are two-dimensional cross-sectional images may be displayed.

### First Embodiment

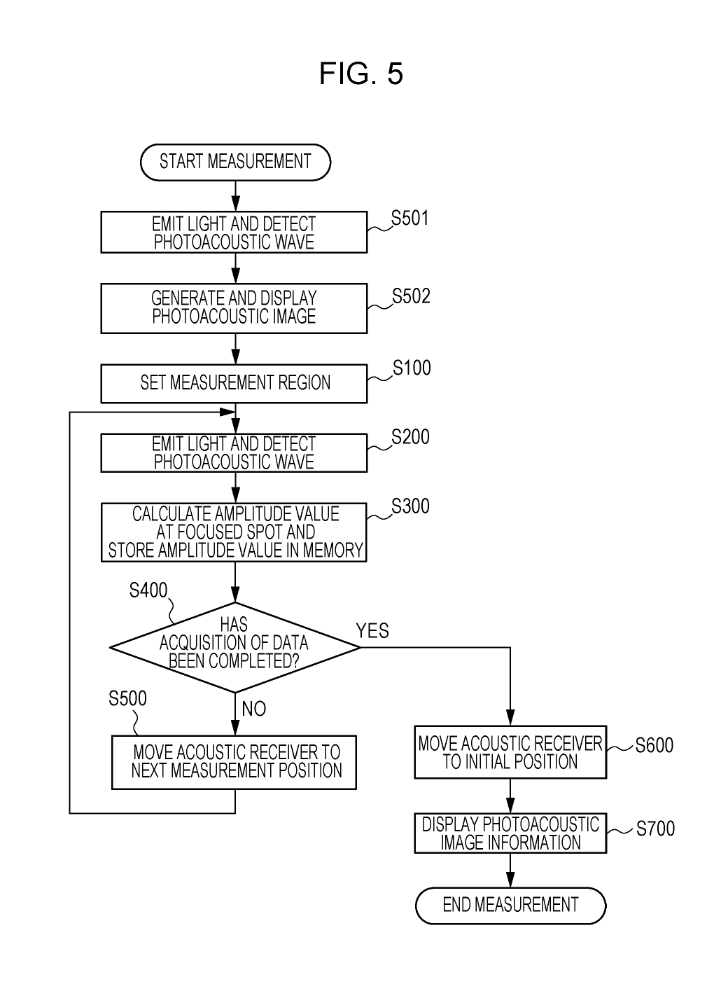

In this embodiment, an acoustic receiver selects a region of interest from a tomography image captured in advance and specifies a scanning region on the basis of the selected region of interest to acquire more detailed photoacoustic image of the region of interest. This process is illustrated in FIG. 5. The process subsequent to step S100 is the same as that illustrated in FIG. 2, and the description thereof will be therefore omitted.

In the acoustic receiver 110, transducers having 256 elements (flat circular plates of Φ 3 mm) are disposed at a bowl-shaped supporting member having the radius of curvature of 5 cm. A scanning pitch is set to 0.1 mm, a scanning region is set to a two-dimensional horizontal region, and the acoustic receiver 110 is disposed so that a focused point becomes flat at the time of the horizontal scanning of the acoustic receiver 110. During acquisition of data by scanning of a probe, a message saying that the scanning of a probe is being performed is displayed on a screen. After a predetermined sequence, a message saying that data acquisition has been completed is displayed on the screen. A measurement target is a scattering phantom in which a light absorber having a radius of 0.5 mm to 1 mm is placed. The light absorber is placed on a horizontal surface on which the focused point of an acoustic receiver is placed.

After the start of a process, in step S501, a photoacoustic wave signal is obtained. The subject 100 is irradiated by the pulsed light 122 emitted from the light source 120 via the optical system 121. The pulsed light 122 is absorbed by the light absorber in the subject 100, so that a photoacoustic wave is generated. Upon detecting the emission of the pulsed light, the control device 140 causes a plurality of transducers in the acoustic receiver 110 to start the reception of a photoacoustic wave. Photoacoustic wave signals output from the acoustic receiver 110 are processed in the control device 140 and are stored in a memory.

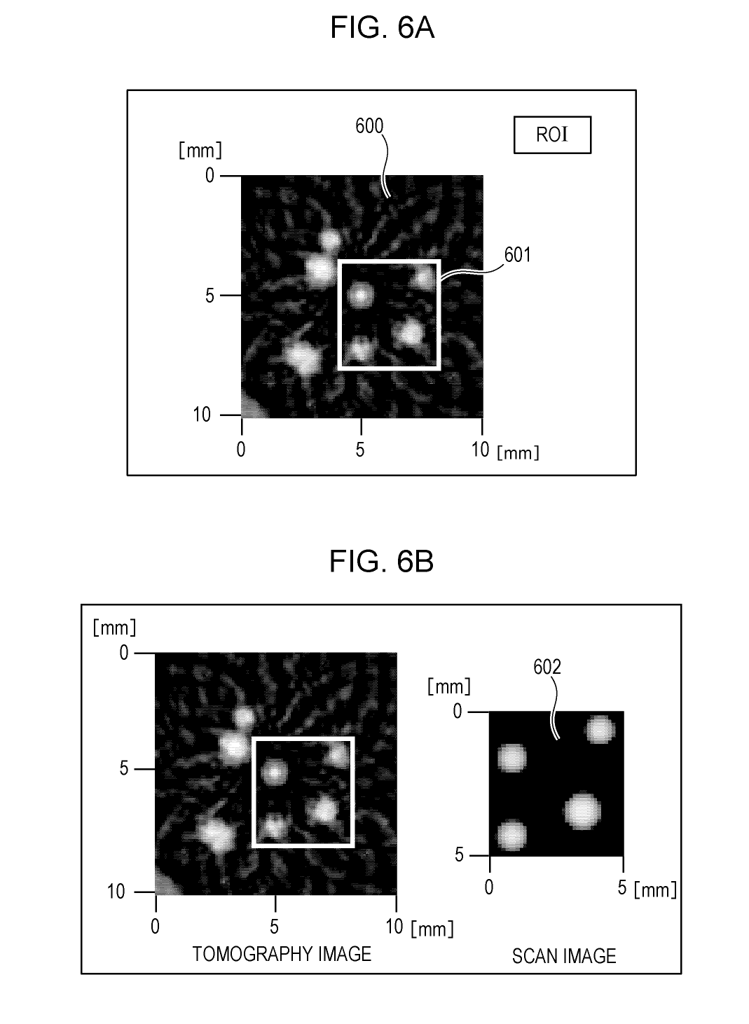

In step S502, the tomography image reconstruction module 152 performs image reconstruction using the photoacoustic wave signals stored in the memory to form characteristic information. The calculated characteristic information is displayed on the display device 160. A display screen at that time is illustrated in FIG. 6A. In a tomography photoacoustic image 600, ball-shaped light absorbers are randomly arranged. However, the shapes of the light absorbers are unclear, and streak signals are observed on the background.

In step S100, a selection region 601 in the tomography photoacoustic image 600 is determined by dragging a mouse. Subsequently, the following process is performed. A screen displayed in step S700 is illustrated in FIG. 6B. The selection region 601 is displayed next to the tomography photoacoustic image obtained in advance as a focused image 602, thereby allowing a user to easily recognize the difference between them. It is apparent from the focused image that the streaks are artifacts. The focused image has a high contrast, and the ratio of the contrast between the background and each light absorber is improved by approximately 25 dB. That is, using this method, the shape of a light absorber can be more precisely reproduced as compared with that in a tomography photoacoustic image. Furthermore, since a tomography photoacoustic image and a more detailed focused image can be formed using a common acoustic receiver, they can be obtained under the same conditions such as a probe size and band characteristics which sometimes have influences on the formation of a photoacoustic image. The configuration of an apparatus can be simplified and there is no need to take a time for exchange of a probe. In this embodiment, the size of the focused image can be smaller than that of the tomography photoacoustic image. That is, it takes a measurement time to form the focused image because each point is scanned. However, since the size of the region of interest is set to a quarter of that of the whole region, the measurement time can be reduced to a quarter of that taken for measurement for the tomography photoacoustic image. Furthermore, a good image with a limited influence of, for example, body motion can be acquired.

### Second Embodiment

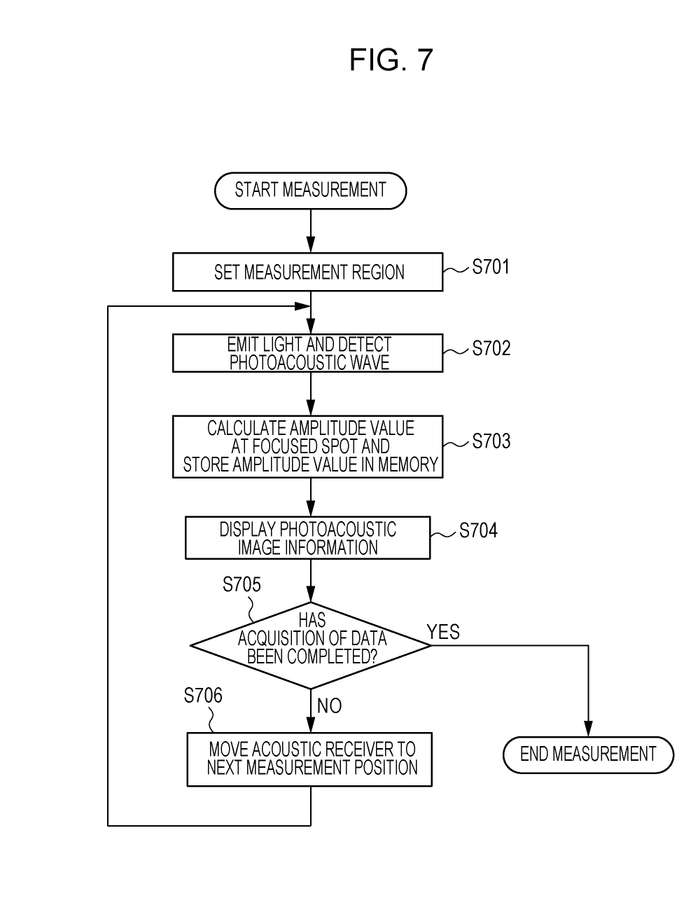

In this embodiment, a method of outputting an image in real-time while allowing an operator to manually move a hand-held probe will be described with reference to FIG. 7.

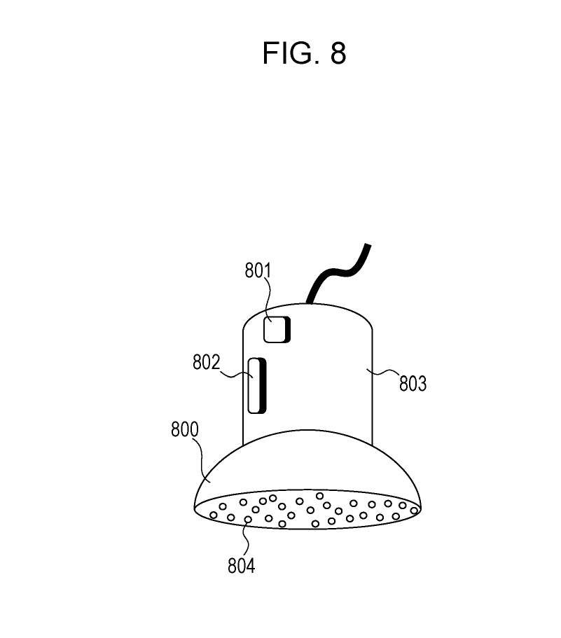

A hand-held probe used in this embodiment will be described with reference to FIG. 8. In a hand-held probe 800, as the acoustic receivers 110, transducers 804 having 256 elements (flat circular plates of Φ 3 mm) are disposed at a bowl-shaped supporting member having the radius of curvature of 5 cm. A focused spot is formed at the same distance from the surface of a bowl in the interior space of the bowl. A gel (not illustrated) having high acoustic consistency is encapsulated in the inside of the bowl so that the acoustic receiver 110 can be brought into contact with a subject on a plane. The probe driving unit 130 and the hand-held probe can be detached from each other. A holding unit 803 is provided to allow an operator to manually perform scanning in a state where the probe driving unit 130 and the hand-held probe are disconnected from each other. A magnetic sensor 802 is attached to the inside of the casing of the holding unit 803. For acquisition of positional information, the control device 140 causes an external receiver to receive the relative positional coordinates and angular information of the probe. A laser repetition frequency is 50 Hz.

During acquisition of data by scanning of a probe, a message saying that the scanning of a probe is being performed is displayed on a screen. After a predetermined sequence, a message saying that data acquisition has been completed is displayed on the screen.

In step S701, like in the first embodiment, in a photoacoustic tomography image obtained in advance, a region of interest is determined. Since a region of interest is manually determined by an operator, there is no need to select a region on a screen like in the first embodiment. However, information about which part of a photoacoustic tomography image, on which a current focused position of the hand-held probe is displayed, a region of interest is placed is displayed with a cursor on the basis of information obtained by the magnetic sensor 802. This allows an operator to accurately recognize a measurement starting position for a focused image.

In step S702, by pressing a photoacoustic signal acquisition switch 801 illustrated in FIG. 8, a photoacoustic signal acquisition sequence is started.

In step S703, processing similar to that of step S300 is performed.

In step S704, a focused photoacoustic image obtained by measurement is superimposed on a photoacoustic tomography image that is a reference image and is then displayed.

In step S705, it is determined whether the photoacoustic signal acquisition switch of the hand-held probe has been touched. It is determined that the switch has not been pressed, measurement ends.

In step S706, processing for moving the probe to the next region of interest while checking a focus cursor icon on a screen is performed.

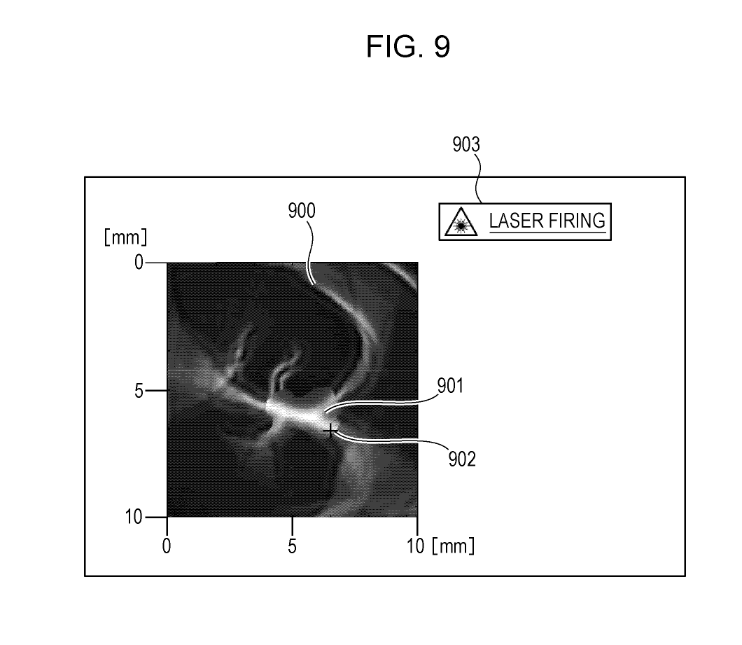

A display screen during measurement is illustrated in FIG. 9. A tomography photoacoustic image 900 that is a reference image is provided. A focused photoacoustic image 901 obtained by updating an image at each focused point is superimposed on the tomography photoacoustic image 900 and is then displayed. As a result, an image part where the number of artifacts has been reduced can be clearly recognized in real time. An image-capturing-in-progress warning icon 903 indicating that measurement is in progress is displayed to alert an operator. At the same time, a focus cursor icon 902 indicating which part of the tomography photoacoustic image 900 a current focused point is present is displayed, thereby allowing an operator to flexibly know the next measurement point.

According to this embodiment, information about a region of interest in a subject can be more freely obtained by operation and an artifact-reduced image can be reproduced in real time.

### Third Embodiment

In this embodiment, an acoustic receiver does not use all of scanning points for scanning of a region of interest. An image is estimated on the basis of a response from a system and scanning points are generated. This method will be described. Using this method, since a scanning time is shortened, a good image with a limited influence of, for example, body motion can be acquired.

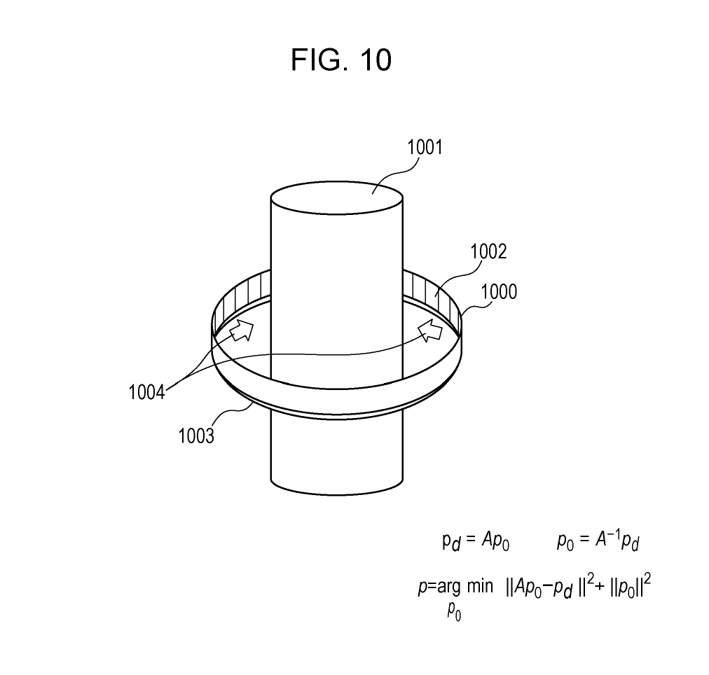

A ring probe used in this embodiment will be described with reference to FIG. 10. In a ring probe 1000, as the acoustic receivers 110, transducers 1002 having 256 elements (having the size of 0.35 mm×7 mm) are disposed at a curved supporting member having the radius of curvature of 1.5 cm. A focused spot is formed at the same distance from the surface of the ring probe in the interior space of a ring. In order to reduce the elevation size of a focused spot and increase a resolution, an acoustic lens is provided on the surface of the probe. A scanning pitch is set to 1 mm, a scanning region is set to a two-dimensional horizontal square region of 1 cm×1 cm, and the ring probe 1000 is disposed so that a focused point becomes flat at the time of the horizontal scanning of the ring probe 1000. Outgoing light 1004 that has been emitted from a light source outputs from a light exit end 1003 via a waveguide and is uniformly applied to a subject 1001. Water is filled between the ring probe 1000 and the subject 1001 for the sake of acoustic consistency at the time of measurement. A light absorber is placed in the subject 1001.

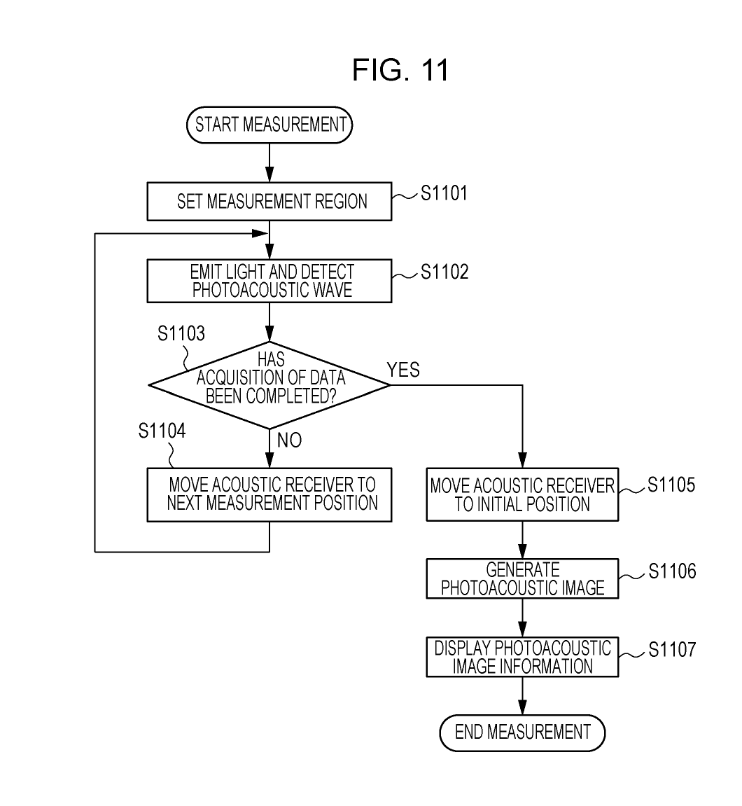

A measurement process is illustrated in FIG. 11. The description of processing similar to that in the above-described embodiments will be omitted.

In step S1101, a square region of 1 cm×1 cm having, at its center, the origin coordinates of the ring probe is set as a region of interest.

In step S1102, the processing of step S200 is performed. In step S1103, the processing of step S400 is performed. In step S1104, the processing of step S500 is performed. In step S1105, the processing of step S600 is performed. In step S1106, the focused image reconstruction module 151 reconstructs the image of the region of interest using photoacoustic wave signals, the summation of which has been performed for each focused spot.

A photoacoustic reception signal Pd can be expressed as a convolution of a sound pressure p₀ generated from a small simple sound source and a spatial response A before reception by a probe (equation 1).

p_{d}=Ap₀ (1)

Accordingly, by obtaining information about the spatial response A in advance, the generated signal p₀ can be estimated (see Japanese Patent Laid-Open No. 2011-143175). The generated sound pressure p is estimated with least squares solution (equation 2). In this equation, however, the constraint of p>0 is added.

$$p = {arg}_{{p}_{0}} min {‖ {Ap}_{0} - {p}_{d} ‖}^{2} + {‖ {p}_{0} ‖}^{2} \qquad ( 2 )$$

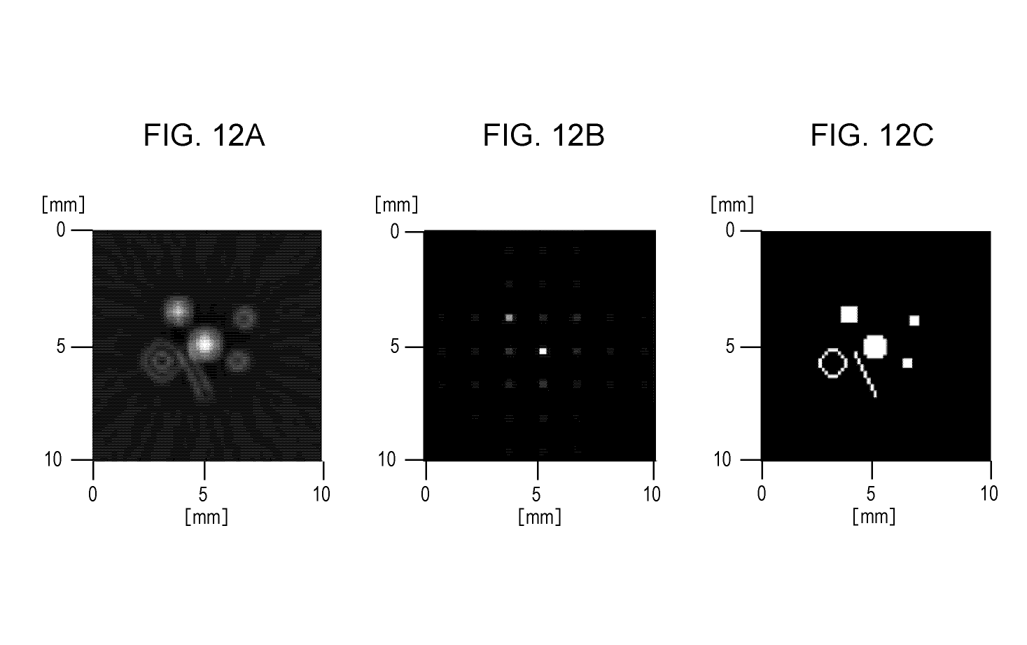

After exhaustive consideration of scanning positions, it has been determined that image reconstruction can be performed without using pieces of data on all scanning points, because spatial responses calculated in advance at scanning positions in proximity to one another are similar to one another. On the basis of pieces of data obtained every 1 mm by scanning, each 0.1-mm-pitch space has been estimated and image reconstruction has been performed with the generated sound pressure p. As a spatial response used, a signal has been generated with a sampling point capable of expressing 0.1 mm. An image reconstructed with this method is illustrated in FIG. 12C. FIG. 12A is a diagram of a comparative example, and illustrates a result of reconstruction of signal data using a tomography method. FIG. 12B illustrates a result of visualization of signals at only focused points among signals at 121 scanning points. Since the number of scanning points is small, it is difficult to grasp an overview. FIG. 12C illustrates a result of image reconstruction according to this embodiment. Although only pieces of information obtained at 121 scanning points are used, image reconstruction is performed using time-series signals. Accordingly, the image appears to nearly exactly reproduce internal conditions.

In step S1107, the final image is displayed through the processing of step S700.

According to this embodiment, the number of scanning points can be reduced and a measurement time can be significantly reduced. More specifically, the number of times of scanning can be reduced to approximately one hundredth part of 10000. In a state where there is a spatial response, a high-contrast image can be obtained. Such image can contribute to the improvement of diagnosis.

According to an embodiment of the present disclosure, even in the case of an apparatus that uses a tomography probe including a plurality of acoustic receivers, it is possible to accurately obtain characteristic information with high contrast and a high signal-to-noise ratio using reception signals obtained at different coordinates corresponding to a plurality of timings, respectively.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of priority from Japanese Patent Application No. 2014-242447 filed Nov. 28, 2014, which is hereby incorporated by reference herein in its entirety.

What is claimed is:

1. A subject information acquisition apparatus comprising:
a light source;
a plurality of receiving units that are arranged along an arc shape and are configured to receive a photoacoustic wave generated in a subject in response to light irradiation from the light source to the subject and convert the photoacoustic wave into a time-series electric signal;
a driving unit configured to scan the plurality of receiving units; and
a processing unit configured to acquire characteristic information about an inside of the subject,
wherein the light source emits light at a certain timing,
wherein the receiving unit receives a photoacoustic wave at a correspondent timing synchronized with emission of light,
wherein the driving unit causes the receiving unit to perform scanning so as to allow the receiving unit to receive the photoacoustic wave in a predetermined region in synchronization with the correspondent timing, and
wherein the processing unit acquires the characteristic information based on positional coordinates in the region and a signal resulting from summation of time-series electric signals corresponding one-to-one to the correspondent timing.

2. The subject information acquisition apparatus according to claim 1, wherein the region is specified by selecting a part of a tomography image of the subject captured in advance.

3. The subject information acquisition apparatus according to claim 1, wherein a reception surface of the receiving unit has a plurality of curvatures.

4. The subject information acquisition apparatus according to claim 3, wherein the processing unit performs summation of signals for each of the plurality of curvatures of the reception surface of the receiving unit.

5. The subject information acquisition apparatus according to claim 1, wherein the receiving unit is disposed on a surface of a supporting member having a curvature.

6. The subject information acquisition apparatus according to claim 1, wherein the processing unit can adjust sound velocities of photoacoustic waves transmitted from a same spatial distance so that electric signals corresponding to the photoacoustic waves are in phase with one another at a time of summation of the electric signals.

7. The subject information acquisition apparatus according to claim 1, wherein the processing unit can execute an image reconstruction algorithm.

8. The subject information acquisition apparatus according to claim 1, wherein the receiving unit is a hand-held receiving unit that is detachable from the driving unit and can be subjected to position control manually performed by an operator.

9. The subject information acquisition apparatus according to claim 8,
wherein the receiving unit includes a magnetic sensor or an optical sensor, and
wherein the processing unit detects relative positional coordinates between the receiving unit and the subject at the correspondent timing based on an output of the magnetic sensor or the optical sensor.

10. The subject information acquisition apparatus according to claim 8, further comprising a detection unit configured to detect positional coordinates in the region.
